(19) United States
(12) Patent Application Publication
(10) Pub. No.: US 2002/0160937 A1
(43) Pub. Date: Oct. 31, 2002

(54) Human vitreous and pharmaceutical compositions

(76) Inventors: Richard Mayne, Birmingham, AL (US); Zhao-Xia Ren, Rockville, MD (US); Jiangang Liu, Fishers, IN (US)

Correspondence Address: Hendricks and Associates, P. O. Box 2509, Fairfax, VA, 22031-2509, US

(21) Appl. No.: 09/801,736

(22) Filed: Mar. 9, 2001

Related U.S. Application Data
(63) Continuation of application No. 09/206,559, filed on Dec. 8, 1998, now abandoned.

(51) Int. Cl. A01N 37/18; A61K 38/00; C07K 1/00; C07K 14/00; C07K 17/00; A61K 35/14; C07K 16/00; A61K 35/16

(52) U.S. Cl. 514/2; 530/381; 424/531

(57) ABSTRACT

In the vitreous, collagen fibrils and hyaluronan molecules form independent networks that interact to maintain the transparency of the vitreous gel. The discovery of the full length cDNA sequence of human vitrin makes it possible to produce the protein and its separate domains by known biotechnological means. It was found that vitrin is released from the collagen fibrils at high salt concentrations. The protein and expressed domains of the invention may be used to facilitate healing of weakened or injured connective tissue.

This application is a continuation of U.S. patent application Ser. No. 09/206,559 filed Dec. 8, 1998, now pending.

This invention was supported by grants from the National Institutes of Health AR 30481 and EY 09908. Hence, the United States Government has certain rights in the invention.

### FIELD OF THE INVENTION

This invention relates to a protein found in the ocular vitreous for use in facilitating the stabilization and healing of connective tissue matrices.

### BACKGROUND OF THE INVENTION

Connective tissue proteins have been known. One such protein known as cartilage matrix protein (CMP) is a noncollagenous protein of the extracellular matrix of cartilage. It is a homotrimer of disulfide bonded subunits. A subunit or analog of cartilage matrix protein which is capable of binding collagen is disclosed and claimed in U.S. Pat. No. 5,686,059, which is incorporated herein by reference in its entirety. Repeats of CMP (called CMP-1 and CMP-2) are homologous to regions within the von Willebrand factor A domain.

The vitreous chamber is the cavity inside the eyeball behind the lens. The vitreous humor or gel is the jelly-like substance contained in the chamber. Vitreous consists of two separate but interactive networks of collagen fibrils and hyaluronan molecules. Complete removal of the hyaluronan network with hyaluronidase or chondroitinase ABC results in the eventual collapse of the gel to form an insoluble aggregate of collagen fibrils. During aging or as a result of trauma or genetically inherited disease, changes occur due to the collapse of the collagen fibrillar network. As the vitreous gel becomes denser, it may contract from the surface of the retina. A similar protein identified as COCH is found in cochlea of ear. Mutations of the latter protein give rise to a form of deafness known as DNFA9.

### SUMMARY OF THE INVENTION

Disclosed herein is the nucleotide and amino acid sequence of human ocular vitreous protein, which has been sequenced and to which polyclonal antibodies have been raised. The vitreous protein, herein referred to as “vitrin”, differs from many other connective tissue proteins in having two von Willebrand A domains. The vitrin has use as an agent to stabilize and facilitate repair of collagen tissues. It also has use as an additive to commercial preparations of hyaluronan which are used for replacling the environment of the vitreous in patients during surgical procedures.

### BRIEF DESCRIPTION OF THE FIGURES

FIG. 1 is the cDNA sequence of human vitrin.

FIG. 2 is a model of the vitrin molecule alone (1) showing the localization of the molecule on the collagen fibrils (2) and in addition interacting with the hyaluronan (3) network.

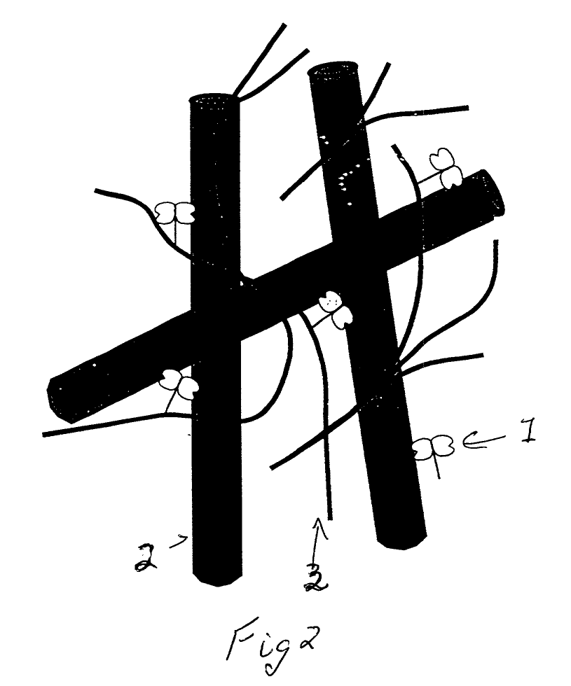

FIG. 3 compares the domains of vitrin with the domain structure of COCH.

FIG. 4 shows each domain of vitrin for which expression was achieved.

FIG. 5 is a photograph of gels showing the domains expressed in bacteria.

### DETAILED DESCRIPTION OF THE INVENTION

In the vitreous, collagen fibrils and hyaluronan molecules form independent networks that interact to maintain the transparency of the vitreous gel. A number of non-collagenous proteins and glycoproteins may contribute to this molecular organization. The new protein of about 70-72 kD was first identified on the surface of collagen fibrils of bovine vitreous. It is soluble in salt solutions, particularly NaCl solutions.

The discovery of the full length cDNA sequence of human vitrin makes it possible to produce the protein and its separate domains by known biotechnological means. This discovery also makes it possible to study the mechanism of collapse of the vitreous gel, which can cause retinal detachment. The instant invention has also made it possible to study the nature of the tractional forces between collapsed collagen fibrils.

It was found that vitrin is released from the collagen fibrils at high salt concentrations. The unique band recognized on SDS-PAGE was subjected to amino acid sequencing of tryptic peptides. All sequences were unique. Cloning of both human and bovine vitrin was achieved using these sequences. Northern blot analysis of vitrin showed that transcripts are present in the extracellular matrix of cardiac muscle, skeletal muscle, kidney, lung and several other tissues. Northern analysis also showed that the transcript for vitrin is about 3.0 kb in size. An in vitro binding assay demonstrated that vitrin is able to bind to collagen fibrils. Hence, it is believed vitrin plays a role in regulating interactions between collagen fibrils and other extra cellular matrix (ECM) components to maintain vitreous integrity. Vitrin also appears to prevent collagen fibrils from aggregating, a process that leads to vitreous gel collapse and posterior vitreous detachment (PVD) with the potential for retinal detachment.

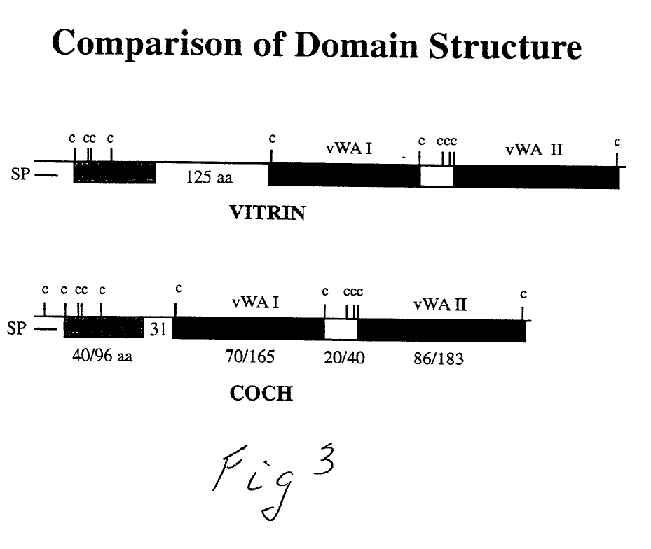

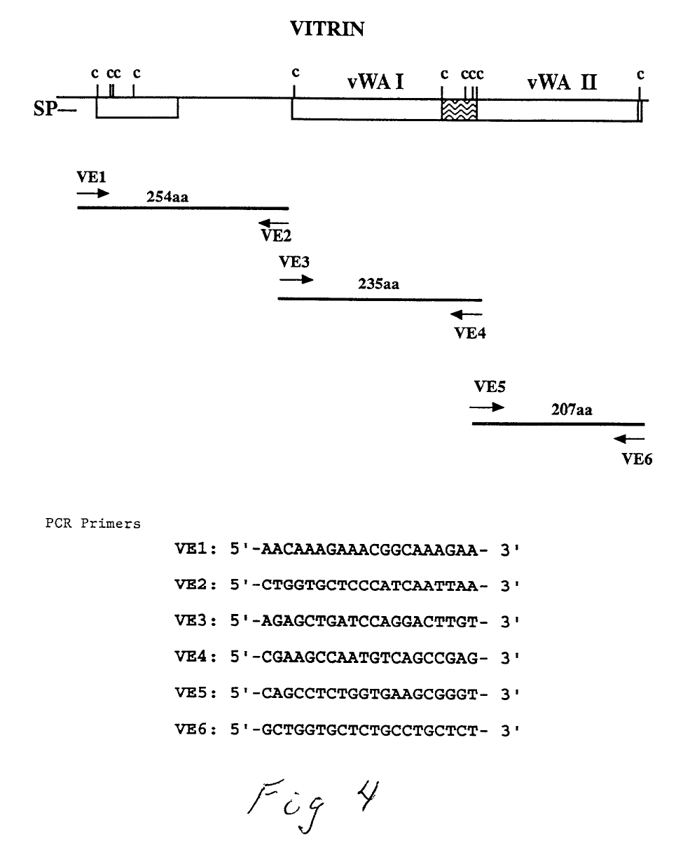

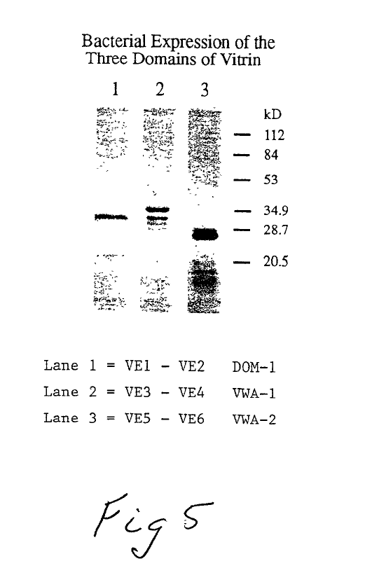

Analysis of the amino acid sequence of vitrin showed the presence of at least four recognizable domains (called DOM-1, vWA1, DOM-3 and vWA2), all of which share the location of cysteine residues with a protein of the cochlea of the ear called COCH (FIG. 3). Recently, the importance of the latter protein was clearly demonstrated by the identification of three mutations in the DOM-1 domain of COCH that give rise to a nonsyndromic form of deafness called DNFA9. For expression of each domain of vitrin, a series of primers were prepared whose location is shown on FIG. 4, resulting in specific amplification by PCR of DOM-1, vWA1 and vWA2. Each PCR product was ligated into PQE30 (Qiagen, Santa Clarita, Calif.) for bacterial expression. (See FIG. 5., which compares the molecular weights of the three domains expressed by bacteria on SDS-PAGE.) Purification of each his-tagged protein was achieved using the protocol provided by the manufacturer (Qiagen). The simplicity of the his-tag purification procedure is preferred, although expression of each domain can also be achieved with a variety of other commercial procedures (PGEX-SX, Pharmacia, Piscataway, N.J., pMAL-C2, New England Biolabs, Beverly, Me.).

The correct folding of the von Willebrand domains can be achieved by bacterial expression systems as is clearly shown by the X-ray crystallographic structure of four von Willebrand A-containing domains, including the I domains of integrins αM, α2, and α1, plus the A3 domain of von Willebrand factor itself. For the latter domain, a his-tag expression system was utilized by both groups demonstrating that this short basic sequence will not interfere with crystallization. Other groups have also achieved expression of I-domains and demonstrated binding to collagen.

Expression of the full-length sequence of vitrin can also be successfully achieved using a baculovirus expression system, as has been successfully achieved for expression of laminin α, β and τ-chains or type III and type I collagen. In addition, successful expression of type III collagen in the yeast Pichia pastoris has been reported. (See U.S. Pat. No. 5,593,859, which is incorporated herein by reference in its entirety.) Several mammalian expression systems have also been utilized to successfully express connective tissue proteins, including type I and II collagen in HT1080 cells, type X procollagen in HEK293 cells, type VI collagen in NIH3T3 cells and type IV collagen in CHO cells. Expression of cartilage matrix protein (CMP) has also been successfully achieved in COS-7 cells and binding of recombinant fragments to collagen shown.

Vitrin was found to consist partly of two von Willebrand A domains which may independently bind to collagen fibrils. The invention makes it possible to study the relationship of vitrin to collagen fibrils and examine vitrin concentration at sites of vitreous collapse.

The cDNA was shown to have the following sequence:

TAGAATAATTTGGATGGGATTTGTGATGCAGGAAAGCCTAAGGGAAAAAGAATATTCATTCTGTG
TGGTGAAAATTTTTTGAAAAAAAAATTGCCTTCTTCAAACAAGGGTGTCATTCTGATATTTATGA
GGACTGTTGTTCTCACTATGAAGGCATCTGTTATTGAAATGTTCCTTGTTTTGCTGGTGACTGGA
GTACATTCAAACAAAGAAACGGCAAAGAAGATTAAAAGGCCCAAGTTCACTGTGCCTCAGATCAA
CTGCGATGTCAAAGCCGGAAAGATCATCGATCCTGAGTTCATTGTGAAATGTCCAGCAGGATGCC
AAGACCCCAAATACCATGTTTATGGCACTGACGTGTATACATCCTACTCCAGTGTGTGTGGCGCT
GCCGTACACAGTAGTGTGCTTGATGATTCAGGAGGGAAAATACTTGTTCGGAAGGTTGCTGGACA
GTCTGGTTACAAAGGGAGTTATTCCAACGGTGTCCAATCGTTATCCCTACCACGATGGAGAGAAT
CCTTTATCGTCTTAGAAAGTAAACCCAAAAAGGGTGTAACCTACCCATCAGCTCTTACATACTCA
TCATCGAAAAGTCCAGCTGCCCAAGCAGGTGAGACCACAAAAGCCTATCAGAGGCCACCTATTCC
AGGGACAACTGCACAGCCGGTCACTCTGATGCAGCTTCTGGCTGTCACTGTAGCTGTGGCCACCC
CCACCACCTTGCCAAGGCCATCCCCTTCTGCTGCTTCTACCACCAGCATCCCCAGACCACAATCA
GTGGGCCACAGGAGCCAGGAGATGGATCTCTGGTCCACTGCCACCTACACAAGCAGCCAAAACAG
GCCCAGAGCTGATCCAGGACTTGTTCCAAAAGAAGAATTGAGCACACAGTCTTTGGAGCCAGTAT
CCCTGGGAGATCCAAACTGCAAAATTGACTTGTCGTTTTTAATTGATGGGAGCACCAGCATTGGC
AAACGGCGATTCCGAATCCAGAAGCAGCTCCTGGCTGATGTTGCCCAAGCTCTTGACATTGGCCC
TGCCGGTCCACTGATGGGTGTTGTCCAGTATGGAGACAACCCTGCTACTCACTTTAACCTCAAGA
CACACACGAATTCTCGAGATCTGAAGACAGCCATAGAGAAAATTACTCAGAGAGGAGGACTTTCT
AATGTAGGTCGGACCATCTCCTTTGTGACCAAGAACTTCTTTTCCAAAGCCAATCGAAACAGAAG
CGGGGCTCCCAATGTGGTGGTGGTGATGGTGGATGGCTGGCCCACGGACAAAGTGGAGGAGGCTT
CAAGACTTGCGAGAGTGTCAGGAATCAACATTTTCTTCATCACCATTGAAGGTGCTGCTGAAAAT
GAGAAGCAGTATGTGGTGGAGCCCAACTTTGCAAACAAGGCCGTGTGCAGAACAAACGGCTTCTA
CTCGCTCCACGTGCAGAGCTGGTTTGGCCTCCACAAGACCCTGCAGCCTCTGGTGAAGCGGGTCT
GCGACACTGACCGCCTGGCCTGCAGCAAGACCTGCTTGAACTCGGCTGACATTGGCTTCGTCATC
GACGGCTCCAGCAGTGTGGGGACGGGCAACTTCCGCACCGTCCTCCAGTTTGTGACCAACCTCAC
CAAAGAGTTTGAGATTTCCGACACGGACACGCGCATCGGGGCCGTGCAGTACACCTACGAACAGC
GGCTGGAGTTTGGGTTCGACAAGTACAGCAGCAAGCCTGACATCCTCAACGCCATCAAGAGGGTG
GGCTACTGGAGTGGTGGCACCAGCACGGGGGCTGCCATCAACTTCGCCCTGGAGCAGCTCTTCAA
GAAGTCCAAGCCCAACAAGAGGAAGTTAATGATCCTCATCACCGACGGGAGGTCCTACGACGACG
TCCGGATCCCAGCCATGGCTGCCCATCTGAAGGGAGTGATCACCTATGCGATAGGCGTTGCCTGG
GCTGCCCAAGAGGAGCTAGAAGTCATTGCCACTCACCCCGCCAGAGACCACTCCTTCTTTGTGGA
CGAGTTTGACAACCTCCATCAGTATGTCCCCAGGATCATCCAGAACATTTGTACAGAGTTCAACT
CACAGCCTCGGAACTGAATTCAGAGCAGGCAGAGCACCAGCAAGTGCTGCTTTACTAACTGACGT
GTTGGACCACCCCACCGCTTAATGGGGCACGCACGGTGCATCAAGTCTTGGGCAGGGCATGGAGA
AACAAATGTCTTGTTATTATTCTTTGCCATCATGCTTTTTCATATTCCAAAACTTGGAGTTACAA
AGATGATCACAAACGTATAGAATGAGCCAAAAGGCTACATCATGTTGAGGGTGCTGGAGATTTTA
CATTTTGACAATTGTTTTCAAAATAAATGTTCGGAATACAGTGCAGCCCTTACGACAGGCTTACG
TAGAGCTTTTGTGAGATTTTTAAGTTGTTATTTCTGATTAGAACTCTGTAACCCTCAGCAAGTTT
CATTTTTGTCATGACAATGTAGGAATTGCTGAATTAAATGTTTAGAAGGATGACAAAAAAAAAAA
AAAAAAAAAAAA

```
!The amino acid sequence of the human virtin was shown to be:
(Seq. #1)
M R T V V L T M K A S V I E M F L V L L V T
G V H S N K E T A K K I K R P K F T V P Q I
N C D V K A G K I I D P E F I V K C P A G C
Q D P K Y H V Y G T D V Y T S Y S S V C G A
A V H S S V L D D S G G K I L V R K V A G Q
S G Y K G S Y S N G V Q S L S L P R W R E S
F I V L E S K P K K G V T Y P S A L T Y S S
S K S P A A Q A G E T T K A Y Q R P P I P G
T T A Q P V T L M Q L L A V T V A V A T P T
T L P R P S P S A A S T T S I P R P Q S V G
H R S Q E M D L W S T A T Y T S S Q N R P R
A D P G L V P K E E L S T Q S L E P V S L G
D P N C K I D L S F L I D G S T S I G K R R
              ▴
F R I Q K Q L L A D V A Q A L D I G P A G P
L M G V V Q Y G D N P A T H F N L K T H T N
S R D L K T A I E K I T Q R G G L S N V G R
T I S F V T K N F F S K A N G N R S G A P N
V V V V M V D G W P T D K V E E A S R L A R
V S G I N I F F I T I E G A A E N E K Q Y V
V E P N F A N K A V C R T N G F Y S L H V Q
                                      ↑
S W F G L H K T L Q P L V K R V C D T D R L
A C S K T C L N S A D I G F V I D G S S S V
                      ▴
G T G N F R T V L Q F V T N L T K E F E I S
D T D T R I G A V Q Y T Y E Q R L E F G F D
K Y S S K P D I L N A I K R V G Y W S G G T
S T G A A I N F A L E Q L F K K S K P N K R
K L M I L I T D G R S Y D D V R I P A M A A
H L K G V I T Y A I G V A W A A Q E E L E V
I A T H P A R D H S F F V D E F D N L H Q Y
V P R I I Q N I C T E F N S Q P R N
                              ↑
```

In the sequence listing above, ▾ designates the start of the von Willebrand domains and ↑ designates the end of the domains.

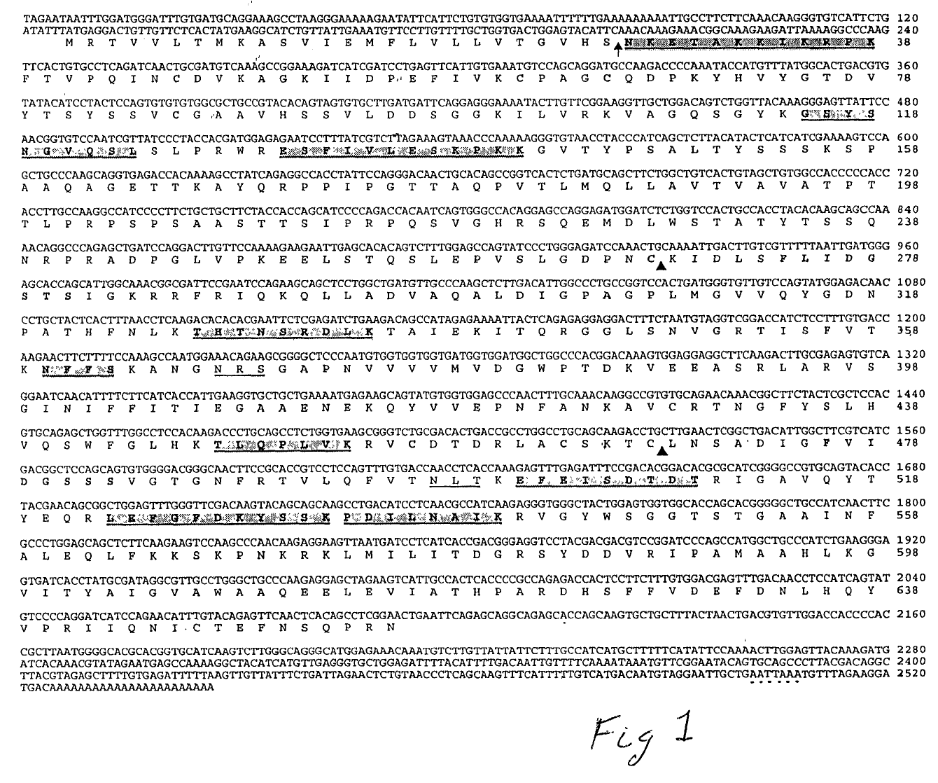

Referring to FIG. 1, the CDNA sequence of human vitrin, ↑ identifies signal peptide cleavage sites. (Hence, ↑ has different meanings in FIG. 1 and in the listing above.) The shaded peptides show the peptides for which amino acid sequence is available.

The ability to raise antibodies to peptide sequences of human vitrin makes it possible to evaluate changes in amount of vitrin in the adult versus the amount in children, to study effects of such changes in disease conditions such as diabetic retinopathy, and to evaluate the amount of vitrin in any given patient tissue. As discussed below, vitrin may also be administered for therapeutic purposes.

Vitrin differs from most other connective tissue proteins in having two von Willebrand A domains which appear to function to crosslink collagen fibrils. The invention, therefore, includes the protein and polypeptides of the protein which are capable of binding collagen. Each von Willebrand domain appears to be primarily involved in the binding of the protein to collagen.

The protein and expressed domains of the invention may be used to facilitate healing of weakened or injured connective tissue. The collagen-binding polypeptides and proteins may be administered to the appropriate sites in need of healing in several ways. It may be applied to the tissue in or on supports such as absorbable supports, including sutures. When the damaged tissue is exposed as, for example, during surgery, the vitrin protein or polypeptides may be applied topically to the tissue. Compositions containing vitrin or collagen-binding polypeptide sequences of human vitrin may be used in high salt solutions. Supports having the inventive proteins or peptides may be placed in the operative area.

Frequently a problem arises when bone or other materials are placed in the patient following breakage or disintegration of bone and connective tissue. The collagen-binding polypeptides and proteins may be applied to prosthetic devices or placed in the region of bone or muscle grafts to enhance growth and development of the connective tissue in the region. The patient's serum may be used as a carrier for the polypeptides and proteins, since it would be a compatible carrier for use in the patient. A concentration of about 0.001% to 5% vitrin is suggested. Whole blood can be drawn from the patient before surgery and the serum collected for use as a carrier for the vitrin. For example, 50 ml of blood is drawn from a patient for purposes of obtaining serum. To the serum obtained from the above sample is added 1 mg of vitrin. The serum containing the vitrin is then applied to a hip prosthesis and the surgical site during surgical implantation.

The partial sequence of the bovine cDNA was deposited in Genbank (number AF063832). The full length human cDNA sequence has been deposited and assigned the number AF 063833.

What we claim is:

1. A purified, isolated polypeptide or protein containing at least one von Willebrand sequence of human vitreous.

2. A polypeptide selected from the group of claim 1 which contains the sequence
K I D L S F L I D G S T S I G K R R F R I Q
K Q L L A D V A Q A L D I G P A G P L M G V
V Q Y G D N P A T H F N L K T H T N S R D L
K T A I E K I T Q R G G L S N V G R T I S F
V T K N F F S K A N G N R S G A P N V V V V
M V D G W P T D K V E E A S R L A R V S G I
N I F F I T I E G A A E N E K Q Y V V E P N
F A N K A V.

3. A polypeptide selected from the group of claim 1 which contains the sequence
L N S A D I G F V I D G S S S V G T G N F R
T V L Q F V T N L T K E F E I S D T D T R I
G A V Q Y T Y E Q R L E F G F D K Y S S K P
D I L N A I K R V G Y W S G G T S T G A A I
N F A L E Q L F K K S K P N K R K L M I L I
T D G R S Y D D V R I P A M A A H L K G V I
T Y A I G V A W A A Q E E L E V I A T H P A
R D H S F F V D E F D N L H Q Y V P R I I Q
N I

4. A composition of matter comprising a polypeptide or protein of claim 1 in an acceptable carrier.

5. A composition of claim 4 wherein the polypeptide selected from the group contains the sequence
K I D L S F L I D G S T S I G K R R F R I Q
K Q L L A D V A Q A L D I G P A G P L M G V
V Q Y G D N P A T H F N L K T H T N S R D L
K T A I E K I T Q R G G L S N V G R T I S F
V T K N F F S K A N G N R S G A P N V V V V
M V D G W P T D K V E E A S R L A R V S G I
N I F F I T I E G A A E N E K Q Y V V E P N
F A N K A V.

6. A composition of claim 4 containing a polypeptide selected from the group which contains the sequence
L N S A D I G F V I D G S S S V G T G N F R
T V L Q F V T N L T K E F E I S D T D T R I
G A V Q Y T Y E Q R L E F G F D K Y S S K P
D I L N A I K R V G Y W S G G T S T G A A I
N F A L E Q L F K K S K P N K R K L M I L I
T D G R S Y D D V R I P A M A A H L K G V I
T Y A I G V A W A A Q E E L E V I A T H P A
R D H S F F V D E F D N L H Q Y V P R I I Q
N I.

7. A composition of claim 4 wherein the carrier is patient serum.
